(19) United States
(12) Patent Application Publication
(10) Pub. No.: US 2014/0303028 A1
(43) Pub. Date: Oct. 9, 2014

(54) GENOMIC DIAGNOSTICS USING CIRCULATING ENDOTHELIAL CELLS

(71) Applicant: Janssen Diagnostics, LLC, Raritan, NJ (US)

(72) Inventors: Yixin Wang, Basking Ridge, NJ (US); Baoying Huang, Lincoln Park, NJ (US); Jack Hu, Hillsborough, NJ (US); Yuqui Jiang, Flemington, NJ (US); Tim Jatkoe, Gladstone, NJ (US); Yadhoda Rajpurohit, Hillsborough, NJ (US)

(21) Appl. No.: 14/313,123

(22) Filed: Jun. 24, 2014

Related U.S. Application Data
(62) Division of application No. 13/540,756, filed on Jul. 3, 2012, now abandoned.

(51) Int. Cl. C12Q 1/68; G01N 33/569

(52) U.S. Cl. CPC C12Q 1/6883; G01N 33/56966
USPC 506/9; 506/16; 435/6.12; 435/6.11

(57) ABSTRACT

Circulating Endothelial Cells are isolated from patient blood and gene expression of the cells is analyzed to assess a medical condition or the tissue of origin of the cell. Kits for conducting the method are also provided.

### BACKGROUND OF THE INVENTION

Circulating endothelial cells (CECs), are present in low number in healthy individuals but an increase of CEC has been observed in a variety of human diseases including cardiovascular disorder and cancer. Characterization of CECs would be beneficial in understanding and monitoring these diseases and others. CECs can be isolated from peripheral blood by a variety of techniques including antibody capture with, for example, CD 146 antibody and magnetic separation as well as flow cytometry and other means. Unfortunately, CEC separation and analysis is complicated by the overwhelming presence of leukocytes. It would be beneficial to identify CECs, relate them to important factors such as their tissues of origin, and provide a basis to further analyze them and provide medical information based upon the analysis.

### SUMMARY OF THE INVENTION

In one aspect of the invention, CECs are isolated and gene expression of CECs is analyzed to assess a medical condition. Preferably, gene expression is conducted using microarray analysis or an amplification and identification method such as reverse transcription PCR (RTPCR).

In another aspect of the invention, genes selected from a group of 130 specific genes whose expression is low in peripheral blood mononuclear cells (PBMCs) and high in endothelial cells are employed as CEC markers.

In yet another aspect of the invention, gene-based CEC markers are those that are associated with one or more of the following: cell motion, cell migration, angiogenesis, or cell adhesion. Preferably, such markers are over-expressed relative to other cells.

In a yet further aspect of the invention, gene-based markers are differentially expressed depending on different vessel types enabling identification of the vessel or tissue of origin of the captured CECs. Preferably, the markers are selected from 67 genes that are over-expressed in CECs relative to other cells.

In a yet further aspect of the invention, cell capture is used to obtain CECs which are analyzed for gene expression. Preferably, capture is via immunomagnetics and the analysis is used to provide a medical assessment such as disease or condition diagnostics, prediction, or prognostics.

In a yet further aspect of the invention, CEC analysis kits are provided. Preferably, the kits include reagents for the identification and analysis of the gene expression of the CECs. Additionally, kits can contain capture reagents for isolating CECs. The kits can also include embodiments of machine code that apply information and algorithms to the information that is produced during the conduct of the CEC isolation and/or analysis such that a medical assessment is produced or facilitated.

### DETAILED DESCRIPTION

The present invention provides compositions, methods and kits for the rapid and efficient isolation and characterization of endothelial cells from biological samples. The methods described isolate and characterize CECs in a blood sample while at the same time minimizing the selection of non-specifically bound or entrapped cells.

While any effective mechanism for isolating, enriching, and analyzing CECs in blood may be used to capture and enrich CECs for analysis, the preferred method for collecting them combines immunomagnetic enrichment technology and immunofluorescent labeling technology with an appropriate analytical platform. The associated tests have the sensitivity and specificity to detect these rare cells in a sample of whole blood and to use them in the analysis of the clinical course of diseases and conditions as well as assessments regarding many aspects of the same including predictions and prognostics.

The capture and separation technology employed in the preferred embodiment is already used widely to analyze circulating tumor cells (CTC) by employing, for example, a tool to investigate the significance of cells of epithelial origin in the peripheral circulation of cancer patients. This technology is described, for example, in U.S. Pat. No. 6,365,362 and U.S. Pat. No. 6,645,731.

The “CellSearch” System (Veridex LLC, Raritan, N.J.) that employs this technology is an automated system based on fluorescence microscopy of isolated cells from blood. It's use in capturing and isolating CECs is also already known. The system contains an integrated computer controlled fluorescence microscope and automated stage with a magnetic yoke assembly that will hold a disposable sample cartridge. The magnetic yoke is designed to enable ferrofluid-labeled candidate rare cells within the sample chamber to be magnetically localized to the upper viewing surface of the sample cartridge for microscopic viewing. Software detects cells labeled with an antibody and having endothelial cells from blood. In a preferred embodiment, a preservative such as “CellSave” cell preservative is used for isolating cells of interest using 7.5 ml of whole blood. Cell-specific magnetic particles are added and incubated, preferably for about 20 minutes. CellSave preservative can be provided in, for example, a tube to the blood sample collector or can be provided as part of the kit of the invention. After magnetic separation, the cells bound to the immunomagnetic-linked antibodies are magnetically held at the wall of the tube. Unbound sample is then aspirated and an isotonic solution is added to resuspend the sample. A nucleic acid dye, monoclonal antibodies to the specified marker and CD 45 (a broad-spectrum leukocyte marker) are incubated with the sample. After magnetic separation, the unbound fraction is again aspirated and the bound and labeled cells are resuspended in 0.2 ml of an isotonic solution. The sample is suspended in a cell presentation chamber and placed in a magnetic device whose field orients the magnetically labeled cells for fluorescence microscopic examination in the CellSearch System. Cells can be identified automatically with control cells enumerated by the System and candidate target cells presented to the operator for checklist enumeration to identify such aspects as morphology. The captured cells can then be subjected to gene-based analysis according to the invention.

Preferred magnetic particles included in the reagents for use in carrying out CEC isolation are particles that behave as colloids. Such particles are characterized by their sub-micron particle size, which is generally less than about 200 nm (0.20 microns), and their stability to gravitational separation from solution for extended periods of time. In addition to the many other advantages, this size range makes them essentially invisible to analytical techniques commonly applied to cell analysis. Particles within the range of 90-150 nm and having between 70-90% magnetic mass are contemplated for use in the present invention. Suitable magnetic particles are composed of a crystalline core of superparamagnetic material surrounded by molecules which are bonded, e.g., physically absorbed or covalently attached, to the magnetic core and which confer stabilizing colloidal properties. The coating material should preferably be applied in an amount effective to prevent non-specific interactions between biological macromolecules found in the sample and the magnetic cores. Such biological macromolecules may include carbohydrates such as sialic acid residues on the surface of non-target cells, lectins, glyproteins, and other membrane components. In addition, the material should contain as much magnetic mass per nanoparticle as possible. The size of the magnetic crystals comprising the core is sufficiently small that they do not contain a complete magnetic domain. The size of the nanoparticles is sufficiently small such that their Brownian energy exceeds their magnetic moment. As a consequence, North Pole, South Pole alignment and subsequent mutual attraction/repulsion of these colloidal magnetic particles does not appear to occur even in moderately strong magnetic fields, contributing to their solution stability. Finally, the magnetic particles should be separable in high magnetic gradient external field separators. That characteristic facilitates sample handling and provides economic advantages over the more complicated internal gradient columns loaded with ferromagnetic beads or steel wool. Magnetic particles having the above-described properties can be prepared by modification of base materials described in U.S. Pat. Nos. 4,795,698, 5,597,531 and 5,698,271.

The immunomagnetic sample preparation is important for reducing sample volume and obtaining a 10⁴ fold enrichment of the target cells. The reagents used in a preferred kit of the invention include: an antibody against the pan-leukocyte antigen, CD45 to identify leucocytes (non-target cells); a cell type specific or nucleic acid dye which allows exclusion of residual red blood cells, platelets and other non-nucleated events; and a biospecific reagent or antibody directed against the target cytostructure or an antibody having specificity for the targets membrane which differs from that used to immunomagnetically select the cells.

Morphological analysis can also be conducted on various analytical platforms and include, for example, the CELLSPOTTER system, a magnetic cell immobilization and analysis system, using microscopic detection for manual observation of cells, described in U.S. Pat. Nos. 5,876,593; 5,985,153 and 6,136,182 respectively. All of the aforementioned U.S. Patent Applications are incorporated by reference herein as disclosing the respective apparatus and methods for manual or automated quantitative and qualitative cell analysis. Other analysis platforms include, but are not limited to, laser scanning Cytometry (Compucyte), bright field base image analysis (Chromavision), and capillary Volumetry (Biometric Imaging).

Kits of the invention preferably include or can be used in conjunction with kits having reagents to conduct a molecular analysis of the cells (CECs) obtained. These include reagents that facilitate methods for determining the gene expression patterns of relevant cells as well as protein based methods of determining gene expression including reverse transcriptase PCR (RT-PCR), competitive RT-PCR, real time RT-PCR, differential display RT-PCR, Northern Blot analysis and other related tests. While it is possible to conduct these techniques using individual PCR reactions, it is best to amplify copy DNA (cDNA) or copy RNA (cRNA) produced from mRNA and analyze it via microarray. A number of different array configurations and methods for their production are known to those of skill in the art and are described in U.S. Patents such as: U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637; the disclosures of which are incorporated herein by reference.

Microarray technology allows for the measurement of the steady-state mRNA level of thousands of genes simultaneously thereby presenting a powerful tool for identifying effects such as the onset, arrest, or modulation of uncontrolled cell proliferation. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in U.S. Pat. No. 6,271,002 to Linsley, et al.; U.S. Pat. No. 6,218,122 to Friend, et al.; U.S. Pat. No. 6,218,114 to Peck, et al.; and U.S. Pat. No. 6,004,755 to Wang, et al., the disclosure of each of which is incorporated herein by reference. Components and reagents for conducting these procedures can be included in the kits of the invention.

In the most preferred embodiments of the invention, patient blood is collected and CECs are isolated using an immunomagnetic capture technique such as is in the CellSearch System. The CECs are then subjected to separate analysis such as with the use of RTPCR or DNA Microarray. Gene expression patterns are determined and processed for the identification of patterns that are indicative of disease diagnosis, prediction, or prognosis which are provided to a clinician and/or the patient. Correlations can be drawn between the expression of the various genes or gene-based markers whose expression is detected and physical conditions such as the presence or absence of disease, disease course or progression, the likelihood of contracting a disease or condition, and other predictive and prognostic judgments. Pattern recognition software can be used to perform these correlations and indicate their presence. Computer instructions that include executable code for comparing assay results with the relevant expression patterns and providing a medical assessment or information useful in providing a medical assessment can be included in the kits of the invention in the form of DVDs, thumbdrives, or any other convenient form.

### EXAMPLES

In the following examples, gene expression profiles of 18 endothelial cell samples from nine anatomical locations were analyzed. A set of 130 gene-based markers with high expression in endothelial cells but not in PBMCs were identified. Detection of these markers from endothelial cells enriched by the CellSearch system was also performed. The gene-based markers were readily detected by QRT-PCR in blood sample spike-in with endothelial cells. Additionally, a set of 67 markers differentially expressed in endothelial cells from different vessel types were identified that can be used to detect the origin of CECs being analyzed.

### Example 1

### CECs

Cryopreserved HAEC (human aorta endothelial cell), HUVEC (human umbilical vein EC), HPAEC (human pulmonary artery EC), HMVEC-ad (human microvascular EC, adult dermis) and HASMC (human artery smooth muscle cell) were obtained from Invitrogen (Invitrogen, Carlsbad, Calif.), cryopreserved HCAEC (human coronary artery EC), HUAEC (human umbilical artery EC), HIAEC (human iliac artery EC), HMVEC-C (Human Cardiac Microvascular EC) cells were obtained from Lonza (Lonza, Cologne, Germany). Cryopreserved HSaVEC (human saphenous vein EC) were obtained from PromoCell (PromoCell GmbH, Heidelberg, Germany). Endothelial cells were cultured with EGM®-2 Endothelial Cell Growth Medium-2 (Lonza) in 37° C. incubator with 95% humidity and 5% CO₂. Smooth muscle cells were cultured with Medium 231 supplemented with Smooth Muscle Growth Supplement (SMGS) (Invitrogen). Cell pellets of HAEC, HUVEC, HPAEC, HMVEC-ad, HCAEC, HUAEC, HMVEC-C, HSaVEC were purchased from PromoCell. Human blood peripheral leukocytes (PBMC) total RNA was purchased from Clontech (Clontech, Mountain View, Calif.).

Blood from healthy donors was drawn into EDTA-containing vacutainer tubes and of which 4 ml was processed by CellSearch system (Veridex LLC, Raritan, N.J.) to isolate CECs. This was done using the CEC Profile kit (Veridex) according to manufacturer's instruction. In spike-in experiments, 500 or 1000 endothelial cells (HAEC, HPAEC, HUVEC or HMVEC-ad) were spiked into 4 ml healthy donor blood and processed by the CellSearch system.

For cultured endothelial cell or cell pellet, about 5×10⁵ cells were used for RNA isolation using AllPrep DNA/RNA Micro Kit (Qiagen, Hilden, Germany). To isolate RNA from CECs enriched by CellSearch System, 350 μl of RLTplus buffer (Qiagen) was added to lyse CECs, then 4 μl poly(I) (Epicentre, Madison, Wis.) of 5 ng/μl was added as carrier RNA, DNA and RNA was isolated using AllPrep DNA/RNA Micro Kit following the manufacturer's instruction. Two samples of the same spike-in was pooled for downstream analysis. The quantity and quality of RNA was examined by NanoDrop 1000 (NanoDrop, Wilmington, Del.) and Agilent Bioanalyzer 2100 (Agilent, Santa Clara, Calif.).

Endothelial cell RNA, smooth muscle cell RNA and PBMC RNA samples were converted into labeled target antisense RNA (cRNA) using the Single-Round RNA Amplification and Enzo Biotin Labeling System. Targets were hybridized to Affymetrix human U133 Plus 2.0 array following protocols as suggested by the supplier (Affymetrix, Santa Clara, Calif.). Following hybridization, arrays were washed and stained using standard Affymetrix procedures before scanning on the Affymetrix GeneChip Scanner and data extraction using Expression Console.

For EC spike-in and donor blood sample processed by CellSearch profile kit, RNA was converted to labeled target cDNA using the Ovation RNA Amplification System V2 (NuGEN, San Carlos, Calif.). Briefly, 50 ng of total RNA was converted to double stranded cDNA using a DNA/RNA chimeric primer for reverse transcription, followed by isothermal amplification. The cDNA was purified using magnetic beads and quantitated by spectrophotometry. 3.75 μg of the purified cDNA subsequently undergoes a two-step fragmentation and labeling process using the Encore Biotin Module (NuGEN). First, the purified cDNA was fragmented to yield single-stranded cDNA products in the 50 to 100 base range. Second, this fragmentation product was labeled via enzymatic attachment of a biotin-labeled nucleotide to the 3-hydroxyl end of the fragmented cDNA generated in the first step. For hybridization, a hybridization cocktail was prepared and added to the fragmentation product using the Hybridization, Wash and Stain kit (Affymetrix), applied to arrays, and incubated at 45° C. for 18 hours. Following hybridization, arrays were washed and stained using standard Affymetrix procedures before scanning on the Affymetrix GeneChip Scanner and data extraction using Expression Console.

Gene expression intensities were extracted with Affymetrix Expression Console (version 1.1) using MASS algorithm. Global scaling was performed to bring the average signal intensity of a chip to a target of 600 before data analysis. To minimize noise levels, probes with fewer than two Presence calls in the cohort were removed. As a result, 31K probe sets remained for subsequent analyses. It was observed that there was a source effect between samples from cell culture and samples from cell pellet. To minimize this effect, probes that showed significant difference (p<0.05) between these two groups were removed. Thus, 23K probes were obtained. To find markers for detecting CECs in the blood, probe sets that had a presence call in the two PBMC samples or had intensity above 200 in either of the two PBMC samples were removed, and 3950 probe sets were retained for further selections.

For hierarchical clustering, signal intensities were normalized to the medium per probe set; hierarchical clustering was performed using Partek Genomics Suites (version 6.5, Partek Inc., St. Louis, Mo.). Hierarchical clustering was conducted on both the probes and the samples using the average linkage method. Euclidean distance metric was used for the calculation of dissimilarity. For unsupervised hierarchical clustering, the 23 k probe sets after removing genes with significant difference between cell cultures and cell pellets were used. Supervised clustering was performed with either 130 CEC markers or 67 skin, artery, and vein EC specific-markers.

Functional annotation was analyzed with the Gene Ontology (GO) classification system using DAVID software (NCIF).

To evaluate the genes selected from the microarray analysis, RNA were extracted from a set of 10 donor samples and 8 cell line spike-in samples (2 HAEC spike-in, 2 HPAEC spike-in, 2 HUVEC spike-in and 2 HMVEC-ad spike-in samples) processed by CellSearch using a CEC Profile kit. 2 μl RNA was used for cDNA synthesis using High-Capacity cDNA Reverse Transcription Kit (Applied Biosystems, Foster City, Calif.) in a 20 μl reaction. To enable multiple gene analysis, 5 μl of cDNA was used to conduct a 14-cycle preamplification using the TaqMan® PreAmp Master Mix Kit (Applied Biosystems) in a 20 μl reaction. The preamplification product was subjected to a 1:20 dilution, and 5 μl of the diluted product was used as input for quantitative PCR. Real time-PCR was carried out using Applied Biosystems gene expression Taqman assays on an ABI PRISM 7900HT Sequence Detector (Applied Biosystems).

### Example 2

### Gene Expression Analysis

RNAs from eighteen endothelial cell samples, two PBMC samples and two smooth muscle cell samples were subjected for microarray analysis using the Affymetrix Human Genome U133 Plus 2.0 Array, which contains more than 54000 probe sets covering 47,000 transcripts and variants, including 38,500 well-characterized human genes. The set of endothelial cell samples represents nine distinct anatomical locations including five different arteries (aorta, coronary artery, pulmonary artery, iliac artery, and umbilical artery), two different veins (umbilical vein and saphenous vein), and two different tissues (skin and heart). Except for iliac artery and skin, two samples including one from cultured cells and one from frozen cell pellet were obtained for each origin. For iliac artery and skin, two samples from the same cultured cells were used and served as technical replicates. To gain an overview of the gene expression pattern, an unsupervised clustering analysis of the gene array data was performed using 31 k probe sets that show present call in at least two samples. Initial analysis of the endothelial cell cluster indicated that the samples from cell culture and samples from cell pellet form different clusters possibly due to difference in sample type. To eliminate this difference, a t-test was performed between these two groups of samples. Probe sets with P-value<0.05 were eliminated resulting in 23 k probe sets. The unsupervised analysis on these 23 k probe sets resulted in three major clusters, the cluster of PBMC samples, the cluster of smooth muscle cell samples and the cluster of endothelial cell samples, reflecting the overall similarity of endothelial cells as compared to PBMC and smooth muscle cells. Within the endothelial cell cluster, the samples from cell pellet and cell culture were not separated, indicating the elimination of source effect. However, samples from same anatomical location were not always cluster together, possibly due to the relative small sample size and the difference of cell culture conditions.

### Example 3

### Use of the CellSearch System to Enrich CECs

The CellSearch system was used to isolate CECs from the spiked donor samples described above. The antibody against the most prominent endothelial membrane antigen CD 146 was used for the immuno-capture reagent. However, there were still about 1000 to 5000 leukocyte cells remaining in the enriched CEC population after the enrichment process. To identify potential CEC specific markers, the expression level of a specific marker in endothelial cells had to be substantially higher than its expression level in PBMC. Thus, probe sets that had a presence call in any of the two PBMC samples or its intensity was above 200 in either of the two PBMC samples were removed. 3950 probes were retained for further analysis. Probes with minimal intensity lower than 1000 in the 18 CEC samples were not considered for subsequent analyses. As a result, 130 probe sets (106 unique genes) were selected as candidates for CEC markers. Functional annotation and pathway analysis was conducted of these 130 probe sets using the DAVID functional annotation software. GO (Gene ontology) term and KEGG (Kyoto Encyclopedia of Genes and Genomes) pathways with significant over-representation are shown in Table 1. The top bioprocess over-represented in these 130 probe sets included genes involved in regulation of cell motion (n=11), regulation of cell migration (n=10) and blood vessel development (n=11). The over-represented cellular component group include plasma membrane (n=44) and focal adhesion (n=6). The over-represented molecular function group include transmembrane receptor protein tyrosine kinase activity (n=7) and protein tyrosine kinase activity (n=8). The major pathways associated with CEC specific genes were focal adhesion (n=12) and ECM-receptor interaction (n=5). One of the functions of endothelial cell is angiogenesis, during which substantial changes in the adhesive interactions between cells and the extracellular matrix (ECM) take place to allow endothelial cell migration.

### Example 3

### Gene-Based Markers

CEC specific genes were identified based on Example 2. These include: Integrin related genes Integrin alpha2 (ITGA2), AXL receptor tyrosine kinase (AXL), EPH receptor A2 (EPHA2), TEK tyrosin kinase, endothelial (TEK), met proto-oncogene (MET), neuropilin 1 (NRP1), VEGFR2 (KDR), and TIE1; angiogenesis related genes activin A receptor type II-like 1 (ACVRL1), connective tissue growth factor (CTGF), endothelial cell-specific chemotaxis regulator (ECSCR), endothelin 1 (EDN1) and roundabout homolog 4 (ROBO4); genes relating to the maintenance of vascular integrity through cell-cell interactions, CAV CAV2, COL4A1, COL5A2, CCND1, FLNB, ITGA2, KDR, LAMA4, LAMB1, PARVA, MET; and independently, MCAM (CD146), KDR (VEGFR-2), TEK (Tie-2). However, some genes associated with endothelial cells are not markers in the present context due to either high expression in PBMC, such as PECAM(CD31), CXCR4, or expression that are too low to be diagnostically useful as seen in one or more endothelial cell lines (such as KIT(SCF R/c-kit) and SELE(E-selectin)). The CEC markers identified in these examples over-represent bioprocess or pathways that are associated with endothelial cell functions.

### Example 4

### Tissue of Origin

The 3950 genes which showed no expression or with intensity less than 200 in both PBMC samples were used to identify gene-based markers useful to identify the tissue of origin of CECs. Artery EC-specific genes were identified as those whose median signal intensity in EC from artery was over 500 and greater than maximum expression in EC from other origins. Likewise, to identify vein EC-specific genes, genes whose median signal intensity in EC from vein was more than 500 and greater than maximum expression in EC from other origins were identified. There were 38 artery EC-specific genes and 14 vein EC-specific genes satisfied these criteria (Table 3). The representative of the artery-specific genes was the previously reported artery EC-specific gene HEY2, a member of the Hairy-related transcription factor family that has been implied to be required for embryonic cardiovascular development in mouse. Other artery specific CEC genes included CXADR; which is a component of tight junction and has been reported to express asymmetrically in heart, in which expression was shown in subendothelial layers of the vessel wall, but not on the luminal endothelial surface. SOX17, a HMG-box transcription factor has been shown to play important roles in both endoderm formation and cardiovascular development, whose promoter activity has been shown in the vascular endothelial cells of arteries in the cardiovascular system but not in veins in a mouse model. To enable the detection of tissue specific markers particularly amenable to use with an immunomagnetic platform such as the CellSearch system, the expression level of the selected markers was significantly higher than that in PBMC. For skin EC-specific genes identification, genes whose minimum expression in EC from skin is 5 fold higher than the maximum expression of all other EC samples were selected. There were 15 such genes.

Supervised clustering was performed on the cell line microarray data using the 67 origin-specific genes. The 67 genes correctly clustered EC from different origin with only one exception (an arterial EC cell clustered with venous EC). Cross verification of the genes was also conducted.

### Example 5

### Verification of Markers by Endothelial Cell Spike-In

Endothelial cells were spiked into healthy donor blood, and then CECs were enriched using the CellSearch System with the CEC Profile kit. Each 4 ml healthy donor blood was spiked in with 500 or 1000 cultured endothelial cells from one origin. Four selected cultured endothelial cell samples including two from artery (HAEC and HPAEC), one from vein (HUVEC), and one from skin (HMVEC-ad) were examined. The enriched CEC samples from spike-in and 10 healthy donor samples without spike-in were subjected to RNA isolation and Microarray analysis. The number of CECs from the 10 donors was determined to be from 2 to 107 in 4 ml blood. Among the 130 CEC specific markers obtained from the cell line gene expression profiling microarray data, all of them shown higher average expression in EC spike-in samples than in donor samples, with the ratio of mean expression in EC spike-in to donor ranging from 2 to 655, and 93 genes have the ratio greater than 20. Validation using QRT-PCR was conducted on 21 markers with a ratio of 8 to 654 between spike-in samples vs. donor samples, and the PCR results demonstrated that all of the selected markers had good separation between EC spike-in samples and non-spike-in donors. To validate the artery-, vein-, and skin-specific markers, a principal component analysis was performed for the spike-in microarray data using the 67 origin-specific markers. Donor samples and artery-, vein-, skin-endothelial cell spike-in samples were clearly separated in the PCA. The markers are thus useful for differentiating CECs from various origins.

### Example 6

### Correlating Disease State with CEC Analysis (Prophetic)

Whole blood is taken from a patient directly into a tube containing CellSave preservative. This is done according to the same protocol used to collect blood for Circulating Tumor Cell (CTC) analysis using the CellSave system. CECs are enriched via the CellSearch system with a CEC cell capture kit. In this system, immunomagnetic enrichment is first conducted with the AUTOPREP separation system to produce an enriched fraction. The kit that is used contains CD146 ferrofluid and reagent to stain the enriched cells with the nucleic acid dye DAPI, endoglin (CD105)-PE and the pan-leukocyte marker CD45APC. The sample is reduced to around 300 uL and placed in an analysis chamber that is mounted inside a magnetic “nest” to magnetically monolayer the cells. Enumeration and morphological analysis of the CEC is then conducted.

CECs are then extracted from the magnetic nesting devices. Total RNA is extracted with the RNeasy Micro Kit (Qiagen, Hilden, Germany). The RNA is converted into cDNA as follows: First, the total amount of extracted RNA is pre-incubated with 300 ng random hexamer at 65° C. for 5 minutes. Then 200 U M-MLV Reverse Transcriptase, RNase H Minus, Point Mutant, M-MLV Reverse Transcriptase 1× Reaction Buffer, 10 U RNasin® Plus RNase Inhibitor (all purchased from Promega, Madison, Wis.), 50 nmol of an equimolarmix of dATP, dTTP, dCTP and dGTP (Amersham Biosciences, Freiburg, Germany) and water is added to a final reaction volume of 20 ul. The reaction is performed at 55° C. for 50 minutes after a pre-incubation step at 20° C. for 10 min. Finally, the reaction is stopped by heating up to 94° C. for 5 min.

Quantitative Reverse-Transcription PCR (qRTPCR) is then performed as follows. Gene expression analysis is conducted duplicate reactions using individual TaqMan® Pre-Developed Assay Reagents specific for the Artery markers in Table 3. In each case they consist of two unlabeled PCR primers and one FAMTM dye-labeled TaqMan® MGB probe as used with the systems described above. The total volume of the reactions is 14 |x1 containing 7 ul 2× TaqMan® Universal PCR Master, 0.7 |x1 TaqMan® Pre-developed Assay Reagents, and 4 |x1 fivefold diluted cDNA template. The PCR amplification is performed using the AB 7900HT Fast Real-time PCR System and consists of an initial incubation at 50° C. for 2 min., then 95° C. for 10 min., followed by 50 cycles of denaturation at 95° C. for 15 s and extension at 60° C. for 1 min. The data are analyzed with the AB7900 Sequence Detection Software version 2.2.2 using automatic baseline correction and cycle threshold setting. Resulting cycle threshold (Ct) data is exported for further analysis. Consumables, equipment and software were purchased from Applied Biosystems, Foster City, Calif., USA.

A number of arterial CEC tissue of origin markers show significant over-expression. The data is downloaded to a file that is used by a program that compares the expression pattern to numerous expression patterns constructed by matching CEC marker expression to known clinical outcomes. The program is contained on storage device that also contains executable code for conducting the comparison using statistically based algorithms. The pattern indicates that the CEC's are arterial in nature and, more specifically, are aortic. Together with other diagnostic information it is concluded that the patient is likely to incur a thoracic aortic aneurism if left untreated.

TABLE 1
Pathway Analysis of Gene Signatures

| | | Number of | |
|---|---|---|---|
| Category | GO Term | genes | P Value |
| Biological | regulation of cell motion | 11 | 2.43E−07 |
| Process | regulation of cell migration | 10 | 8.03E−07 |
| | blood vessel development | 11 | 2.15E−06 |
| | regulation of locomotion | 10 | 2.32E−06 |
| | vasculature development | 11 | 2.68E−06 |
| | blood vessel morphogenesis | 10 | 5.04E−06 |
| | angiogenesis | 8 | 3.12E−05 |
| | cytoskeleton organization | 11 | 2.93E−04 |
| | tube development | 8 | 3.70E−04 |
| | regulation of response to external | 7 | 4.02E−04 |
| | stimulus | | |
| | transmembrane receptor protein | 8 | 4.12E−04 |
| | tyrosine kinase signaling | | |
| | pathway | | |
| Cellular | plasma membrane | 44 | 2.47E−05 |
| Component | focal adhesion | 6 | 5.50E−04 |
| | plasma membrane part | 28 | 6.12E−04 |
| | cell-substrate adherens junction | 6 | 6.55E−04 |
| | intrinsic to plasma membrane | 19 | 7.74E−04 |
| | cell-substrate junction | 6 | 8.41E−04 |
| | extracellular matrix part | 6 | 1.02E−03 |
| Molecular | transmembrane receptor protein | 7 | 3.35E−06 |
| Function | tyrosine kinase activity | | |
| | protein tyrosine kinase activity | 8 | 7.14E−05 |
| KEGG | Focal adhesion | 12 | 7.07E−08 |
| PATHWAY | ECM-receptor interaction | 5 | 2.73E−03 |

TABLE 2
Identified CEC Gene-Based Markers

| | Gene | |
|---|---|---|
| Affymetrix ID | Symbol | Accession |
| 1555233_at | RHOJ | BC025770 |
| 1556037_s_at | HHIP | AK098525 |
| 200756_x_at | CALU | U67280 |
| 200832_s_at | SCD | AB032261 |
| 201289_at | CYR61 | NM_001554 |
| 201325_s_at | EMP1 | NM_001423 |
| 201431_s_at | DPYSL3 | NM_001387 |
| 201445_at | CNN3 | NM_001839 |
| 201467_s_at | NQO1 | AI039874 |
| 201616_s_at | CALD1 | AL577531 |
| 201785_at | RNASE1 | NM_002933 |
| 201801_s_at | SLC29A1 | AF079117 |
| 201843_s_at | EFEMP1 | NM_004105 |
| 202052_s_at | RAI14 | NM_015577 |
| 202134_s_at | WWTR1 | NM_015472 |
| 202237_at | NNMT | NM_006169 |
| 202238_s_at | NNMT | NM_006169 |
| 202619_s_at | PLOD2 | AI754404 |
| 202620_s_at | PLOD2 | NM_000935 |
| 202628_s_at | SERPINE1 | NM_000602 |
| 202686_s_at | AXL | NM_021913 |
| 202733_at | P4HA2 | NM_004199 |
| 202766_s_at | FBN1 | NM_000138 |
| 202976_s_at | RHOBTB3 | NM_014899 |
| 202998_s_at | LOXL2 | NM_002318 |
| 203002_at | AMOTL2 | NM_016201 |
| 203323_at | CAV2 | BF197655 |
| 203324_s_at | CAV2 | NM_001233 |
| 203499_at | EPHA2 | NM_004431 |
| 203510_at | MET | BG170541 |
| 203811_s_at | DNAJB4 | NM_007034 |
| 203934_at | KDR | NM_002253 |
| 204135_at | FILIP1L | NM_014890 |
| 204248_at | GNA11 | NM_002067 |
| 204281_at | TEAD4 | NM_003213 |
| 204337_at | RGS4 | AL514445 |
| 204338_s_at | RGS4 | NM_005613 |
| 204339_s_at | RGS4 | BC000737 |
| 204468_s_at | TIE1 | NM_005424 |
| 204517_at | PPIC | BE962749 |
| 204602_at | DKK1 | NM_012242 |
| 204975_at | EMP2 | NM_001424 |
| 205120_s_at | SGCB | U29586 |
| 205573_s_at | SNX7 | NM_015976 |
| 205618_at | PRRG1 | NM_000950 |
| 206331_at | CALCRL | NM_005795 |
| 206702_at | TEK | NM_000459 |
| 207469_s_at | PIR | NM_003662 |
| 207714_s_at | SERPINH1 | NM_004353 |
| 208025_s_at | HMGA2 | NM_003483 |
| 208613_s_at | FLNB | AV712733 |
| 208712_at | CCND1 | M73554 |
| 208789_at | PTRF | BC004295 |
| 208790_s_at | PTRF | AF312393 |
| 209094_at | DDAH1 | AL078459 |
| 209101_at | CTGF | M92934 |
| 209109_s_at | TSPAN6 | U84895 |
| 209120_at | NR2F2 | AL037401 |
| 209387_s_at | TM4SF1 | M90657 |
| 209487_at | RBPMS | D84109 |
| 209488_s_at | RBPMS | D84109 |
| 209676_at | TFPI | J03225 |
| 210041_s_at | PGM3 | BC001258 |
| 210089_s_at | LAMA4 | BC004241 |
| 210762_s_at | DLC1 | AF026219 |
| 210764_s_at | CYR61 | AF003114 |
| 210815_s_at | CALCRL | U17473 |
| 210933_s_at | FSCN1 | BC004908 |
| 211340_s_at | MCAM | M28882 |
| 211564_s_at | PDLIM4 | BC003096 |
| 211651_s_at | LAMB1 | M20206 |
| 211980_at | COL4A1 | AI922605 |
| 212093_s_at | MTUS1 | AI695017 |
| 212095_s_at | MTUS1 | BE552421 |
| 212097_at | CAV1 | AU147399 |
| 212104_s_at | RBM9 | N95026 |
| 212298_at | NRP1 | BE620457 |
| 212985_at | APBB2 | BF115739 |
| 212992_at | AHNAK2 | AI935123 |
| 213010_at | PRKCDBP | AI088622 |
| 213306_at | MPDZ | AA917899 |
| 213901_x_at | RBM9 | AW149379 |
| 217553_at | MGC87042 | AW129021 |
| 217820_s_at | ENAH | NM_018212 |
| 217890_s_at | PARVA | NM_018222 |
| 218665_at | FZD4 | NM_012193 |
| 218678_at | NES | NM_000950 |
| 218736_s_at | PALMD | NM_017734 |
| 218995_s_at | EDN1 | NM_001955 |
| 219522_at | FJX1 | NM_014344 |
| 220027_s_at | RASIP1 | NM_017805 |
| 221730_at | COL5A2 | NM_000393 |
| 222433_at | ENAH | AK025108 |
| 222454_s_at | PARVA | BG107577 |
| 223279_s_at | UACA | AF322916 |
| 223315_at | NTN4 | AF278532 |
| 223775_at | HHIP | AY009951 |
| 224822_at | DLC1 | AA524250 |
| 224894_at | YAP1 | BF247906 |
| 225162_at | SH3D19 | BG285417 |
| 225163_at | FRMD4A | BF000162 |
| 225464_at | FRMD6 | N30138 |
| 225481_at | FRMD6 | AL040051 |
| 226028_at | ROBO4 | AA156022 |
| 226084_at | MAP1B | AA554833 |
| 226302_at | ATP8B1 | BG290908 |
| 226751_at | CNRIP1 | AW193693 |
| 226950_at | ACVRL1 | T63524 |
| 227314_at | ITGA2 | N95414 |
| 227529_s_at | AKAP12 | BF511276 |
| 227628_at | GPX8 | AL571557 |
| 228141_at | GPX8 | AA173223 |
| 228158_at | LOC645166 | AI623211 |
| 228297_at | SLIT2 | AI807004 |
| 228339_at | ECSCR | AA181256 |
| 228748_at | CD59 | AI653117 |
| 228824_s_at | PTGR1 | BE566894 |
| 230250_at | PTPRB | AI670852 |
| 231094_s_at | MTHFD1L | AL035086 |
| 231319_x_at | KIF9 | AI657069 |
| 231897_at | PTGR1 | AL135787 |
| 233660_at | EHD4 | BG540685 |
| 235391_at | FAM92A1 | AW960748 |
| 235489_at | RHOJ | AI583530 |
| 236565_s_at | LARP6 | BF792126 |
| 236656_s_at | LOC100288911 | AW014647 |
| 237466_s_at | HHIP | AW444502 |
| 238905_at | RHOJ | BE218803 |
| 238906_s_at | RHOJ | BE218803 |
| 242321_at | PTPN14 | AI628689 |

TABLE 3
Tissue Specific Gene-Based Markers
Artery EC markers

| Affymetrix ID | Gene Symbol | Accession | Affymetrix ID | Gene Symbol | Accession |
|---|---|---|---|---|---|
| 201430_s_at | DPYSL3 | W72516 | 222486_s_at | ADAMTS1 | AF060152 |
| 201431_s_at | DPYSL3 | NM_001387 | 222921_s_at | HEY2 | AF232238 |
| 204518_s_at | PPIC | NM_000943 | 225303_at | KIRREL | AI049973 |
| 204944_at | PTPRG | NM_002841 | 226374_at | CXADR | BG260087 |
| 204948_s_at | FST | NM_013409 | 226847_at | FST | BF438173 |
| 205226_at | PDGFRL | NM_006207 | 227623_at | CACNA2D1 | H16409 |
| 205422_s_at | ITGBL1 | NM_004791 | 228011_at | FAM92A1 | BF338870 |
| 206832_s_at | SEMA3F | NM_004186 | 228507_at | CNN3 | AI742043 |
| 209730_at | SEMA3F | U38276 | 228640_at | PCDH7 | BE644809 |
| 209897_s_at | SLIT2 | AF055585 | 228850_s_at | PDE3A | AI963304 |
| 209990_s_at | GABBR2 | AF056085 | 229715_at | DKFZp686O24166 | AW006182 |
| 211679_x_at | GABBR2 | AF095784 | 230112_at | MARCH4 | AB037820 |
| 214927_at | ITGBL1 | AL359052 | 231361_at | NLGN1 | AI912122 |
| 217077_s_at | GABBR2 | AF095723 | 235228_at | CCDC85A | AI376433 |
| 218665_at | FZD4 | NM_012193 | 235391_at | FAM92A1 | AW960748 |
| 219249_s_at | FKBP10 | NM_021939 | 240770_at | TMEM171 | AW058459 |
| 219743_at | HEY2 | NM_012259 | 242162_at | WDR69 | AA904430 |
| 219993_at | SOX17 | NM_022454 | 1555240_s_at | GNG12 | AF493879 |
| 222162_s_at | ADAMTS1 | AK023795 | 1557080_s_at | ITGBL1 | AI753143 |

| Vein EC markers | | | Skin EC markers | | |
|---|---|---|---|---|---|
| Affymetrix ID | Gene Symbol | ACCESION | Affymetrix ID | Gene Symbol | ACCESION |
| 202052_s_at | RAI14 | NM_015577 | 203000_at | STMN2 | BF967657 |
| 205923_at | RELN | NM_005045 | 204879_at | PDPN | NM_006474 |
| 209120_at | NR2F2 | AL037401 | 205515_at | PRSS12 | NM_003619 |
| 210089_s_at | LAMA4 | BC004241 | 205743_at | STAC | NM_003149 |
| 210990_s_at | LAMA4 | U77706 | 211959_at | IGFBP5 | AW007532 |
| 211538_s_at | HSPA2 | U56725 | 213802_at | PRSS12 | AI810767 |
| 218816_at | LRRC1 | NM_018214 | 218468_s_at | GREM1 | AF154054 |
| 221371_at | TNFSF18 | NM_005092 | 218469_at | GREM1 | NM_013372 |
| 221880_s_at | FAM174B | AI279819 | 221898_at | PDPN | AU154455 |
| 228837_at | TCF4 | BE857360 | 226658_at | PDPN | AW590196 |
| 51158_at | FAM174B | AI801973 | 228716_at | THRB | BG494007 |
| 228840_at | AMOTL1 | AW451115 | 228875_at | FAM162B | AI540210 |
| 230192_at | TRIM13 | AI472310 | 236420_s_at | ANO4 | BF589515 |
| 1555564_a_at | CFI | BC020718 | 237056_at | INSC | BF432206 |
| | | | 1552445_a_at | ESX1 | NM_153448 |

What is claimed is:

1. A method of assessing medical condition comprising identifying differential expression of gene-based markers (relative to the expression of the same genes in a normal population) in CECs.

2. The method of claim 1 wherein the markers are not differentially expressed in PBMCs.

3. The method of claim 1 wherein there is at least a 2 fold difference in the expression of the modulated genes.

4. The method of claim 1 wherein the p-value indicating differential modulation is less than 0.05.

5. The method of claim 1 wherein the markers are selected from Table 2.

6. A method of identifying the tissue of origin of a CEC comprising identifying differential expression of tissue specific gene-based markers (relative to the expression of the same genes in a normal population) in CECs.

7. The method of claim 6 wherein the markers are not differentially expressed in PBMCs.

8. The method of claim 1 wherein there is at least a 2 fold difference in the expression of the modulated genes.

9. The method of claim 1 wherein the p-value indicating differential modulation is less than 0.05.

10. The method of claim 1 wherein the markers are selected from Table 3.

11. The method of claim 4 wherein there is at least a 2 fold difference in the expression of the modulated genes.

12. The method of claim 4 wherein the p-value indicating differential modulation is less than 0.05.

13. A diagnostic kit comprising reagents for isolating CECs and reagents for detecting the expression of the markers of Table 2.

14. The diagnostic kit of claim 13 wherein the reagents for detecting markers are those of Table 3.

15. The kit of claim 13 further comprising reagents for conducting a microarray analysis.

16. The kit of claim 13 further comprising reagents for amplifying the markers.

17. The kit of claim 13 further comprising a component containing executable instructions that correlate expression detection with a pattern.

18. The kit of claim 13 wherein said reagents for isolating CECs include immunomagnetic reagents.

19. A method for assessing medical condition comprising isolating CECs, amplifying gene expression markers from said CECs, detecting the expression of said markers, and correlating the expression of said markers with a diagnosis or prognosis; wherein isolation and amplification is conducted using the kit of claim 13.

20. A method for assessing medical condition comprising isolating CECs, amplifying gene expression markers from said CECs, detecting the expression of said markers, and correlating the expression of said markers with a diagnosis or prognosis; wherein isolation and amplification is conducted using the kit of claim 14.
